(19) United States
(12) Patent Application Publication
(10) Pub. No.: US 2014/0323802 A1
(43) Pub. Date: Oct. 30, 2014

(54) Methods and Systems for Improving the Prevention of Colorectal Cancer

(71) Applicant: Stephen C. Lloyd, Columbia, SC (US)

(72) Inventor: Stephen C. Lloyd, Columbia, SC (US)

(21) Appl. No.: 14/203,031

(22) Filed: Mar. 10, 2014

Related U.S. Application Data
(60) Provisional application No. 61/774,972, filed on Mar. 8, 2013.

(51) Int. Cl. A61B 1/31; A61B 10/04; A61B 17/22; G09B 23/28

(52) U.S. Cl. CPC A61B 1/31; G09B 23/285; A61B 10/04; A61B 17/22031
USPC 600/104; 434/272; 600/562; 600/109; 705/2

(57) ABSTRACT

A method for improving the prevention of colorectal cancer in a patient, comprising: conducting a colonoscopy which includes a first medical professional navigating a colonoscope in a colon of the patient and, simultaneously, a second medical professional controlling a tip of the colonoscope to perform at least one medical procedure.

### RELATED APPLICATIONS

This application claims the benefit of priority under 35 U.S.C. §119(e) of U.S. Provisional Patent Application No. 61/774,972 filed Mar. 8, 2013, the contents of which are incorporated by reference herein in their entirety.

### FIELD AND BACKGROUND OF THE INVENTION

The present invention, in some embodiments thereof, relates to cancer prevention and, more particularly, but not exclusively, to methods and systems for improving colorectal cancer prevention.

Colorectal cancer (“CRC”) is the #1 Cancer Killer among non-smokers in the United States. CRC is the second leading cause of cancer-related deaths in the U.S. Over 150,000 Americans are diagnosed with colorectal cancer each year and over 50,000 die, this represents 3% of all deaths annually. The lifetime risk of developing colorectal cancer in the United States is one in twenty. Ninety percent of new cases and 95% of deaths occur in people 50 years of age or older. Direct medical costs for treatment for a patient with advanced (stage III or IV) CRC average $360,000 per person. Unfortunately, over half of patients diagnosed with CRC are detected at advanced stages after symptoms occur.

CRC is unique among common deadly cancers in that there is a precursor: the polyp. Through a process referred to as the “polyp-to-cancer sequence” a polyp slowly evolves into cancer. This evolution typically takes longer than a decade. This long window provides a golden opportunity to interrupt this deadly progression through the identification and destruction of these pre-malignant lesions. Removal of pre-cancerous polyps is an effective mechanism of preventing colorectal cancer. The initial identification of this sequence is the scientific foundation upon which the recommendation for colonoscopy is based. The early research (the 1995 National Polyp Study) predicted an eighty-four percent reduction in colorectal cancer cases with screening colonoscopy for all Americans beginning at age fifty and repeated every ten years. Unfortunately, realization of this dream has eluded us. In contrast to the outstanding protection from subsequent colorectal cancer predicted by these studies, Americans continue to contract, and die from, colorectal cancer at alarming rates. Two obstacles remain impeding realization of the dream: compliance and quality. Far too few Americans take advantage of the benefits afforded from colonoscopy screening and the protective benefits from colonoscopy appear strikingly non-uniform.

Ninety percent of CRC patients survive over five years if discovered early in the course of the disease when restricted within the muscular walls of the colon, where it has not spread to other areas. If the cancer has spread to other organs, survivability drops to only 12 percent. Discovered early, the medical costs related to removal and follow-up are minimal compared to that which will be spent on patients with CRC in the “distant” or later stages.

While CRC screening done to the highest standard of care virtually eliminates the possibility of CRC (90 percent prevention), only 35 percent of those eligible to get colonoscopies do so, and the quality of screening varies unacceptably among those. Persons 50 and older in the U.S. total over 120 million, and represent the fastest growing age segment of the U.S. population. Over 6 million of these people will ultimately develop colorectal cancer. Assuming 35% still get colonoscopies (the current rate), and those procedures are all of the highest standard of care resulting in prevention (or at worst earlier detection) and minimal related costs, that leaves 65% not getting the procedure at current rates that will evolve into full blown CRC. With current cost per patient with colorectal cancer at $360,000 cost in current dollars, $780 billion dollars in medical costs for colorectal cancer will be spent to care for these 3.9 million people. Approximately $400 billion of that cost is unnecessary because advanced CRC cases could have been prevented with a high quality colonoscopy screening. Related costs, or so called “indirect costs” including losses to the workforce, family disruption, and so forth, add significant additional unnecessary costs reaching or exceeding a trillion dollars in total expenditures that would not be incurred if colonoscopy screening compliance was equivalent to the compliance level currently achieved for breast cancer screening (seventy-five percent compliance).

Colonoscopy is a medical procedure whereby a flexible fiber optic instrument is introduced into the rectum and advanced through the colon (i.e. large intestine) for detailed examination of the colonic surface. The colon is a hollow, muscular tube or lumen about 6 feet long, consisting of six parts or segments: beginning with the cecum (where the appendix attaches), then on to the ascending colon, transverse colon, descending colon, sigmoid and terminating with the rectum. Colonoscopy allows for inspection of the entire colon and provides the ability to perform a number of therapeutic operations such as biopsy and polyp removal during a single procedure. A colonoscopy procedure consists of two phases: an insertion phase and a withdrawal phase. During the insertion phase, a flexible endoscope is advanced under direct vision via the anus into the rectum and then gradually into the most proximal part of the colon, the cecum. In the withdrawal phase, the endoscope is gradually withdrawn while the specialist moves the camera in a circular or circumferential fashion to carefully examine the entire surface inspecting for suspicious lesions. Traditionally, the purpose of the insertion phase is to expeditiously (while patient is still sedated) reach the cecum, which represents the end of the large intestine. Careful mucosa inspection and diagnostic or therapeutic interventions such as biopsy, polyp removal, etc., are currently performed during the withdrawal phase.

Colonoscopy is the “Gold Standard” procedure for screening for colorectal cancer. Why is this basic medical procedure not going to be taken by 3.9 million future CRC patients currently among the current over 50-year old population U.S. population? Mammograms are taken by 75% of the women in this population, and an even larger rate among men getting prostate exams. Why do most of them still not get a quality colonoscopy? Test for test, a high quality colonoscopy is fifty times more likely to save a woman's life than a mammogram. Most of them know the risk of CRC. Most of them know the procedure can detect it CRC early and, if so, prevent death if found early enough. Still, they elect not to have the procedure done.

Number of lives that could be saved if we mirrored breast cancer screening compliance: 40,000/year. Cost-new chemotherapeutic interventions often cost over $50,000 per patient and result in 2.1 months of life extension. As a result, treatment of advanced colorectal cancer averages $360,000 per patient.

Creating a method that overcomes compliance and quality issues will provide an unprecedented standard of care in pre-cancerous identification, removal, and testing which would promise to dramatically reduce the number of those 3.9 million Americans that will otherwise die of colorectal cancer. That method will not only save those lives, but it will add more productivity and quality of life to those lives and their families, friends and associates. Finally, it will save a large portion of the trillion dollars otherwise spent for the cost of caring for those CRC patients and the time/cost/trouble expended by their families and caregivers.

Additional background art includes non-patent literature:
1. Harewood G C, Sharma V K. Garmo P: Impact of colonoscopy preparation quality on detection of suspected colonic neoplasia. Gastrointest Endosco 2003: 58(1):76-79;
2. Kim H N, Raju G S. Bowel preparation and colonoscopy technique to detect non-polypoid colorectal neoplasms. Gastrointest Endosco Clin N Am. 2010 July: 20(3): 437-448;
3. Abuksis G., Mor M., Segal N., et al. A patient education program is cost effective for preventing failure of endoscopic procedures in a gastroenterology department. Am J Gastroenterol 2001; 96(6): 1786-90;
4. Froehlich F, Wietlisbach V, Gonvers J J, et al. Impact of colonic cleansing on quality and diagnostic yield of colonoscopy: The European Panel on Appropriateness of Gastroinstestinal Endoscopy European MultiCenter Study. Gastrointest Endosc 2005:61(3):378-84;
5. Sanaka M R. Deepinder F, Thota P N, Lopez R, Burke, C A. Adenomas are detected more often in morning than in afternoon colonoscopy. Am J Gastroenterol 2009;104:1659-64;
6. United States Preventive Services Task Force 2008. Screening for colorectal cancer. Release date October 2008. Accessed Nov. 20, 2012 at www.uspreventiveservicestaskforce.org/uspstf/uspscolo.htm;
7. Williams A R, Balasooriya B A, Day D W. Polyps and cancer of the large bowel: a necropsy study in Liverpool. Gut 1982:23:835-42; and,
8. Lee Y S. Early malignant lesions of the colorectum at autopsy. Dis Colon Rectum 31(4):291-97.

### SUMMARY OF THE INVENTION

An aspect of some embodiments of the invention relates to methods for improving the prevention of colorectal cancer including one, some, or all of the following features: a) two-person colonoscope control technique; b) at least three or four persons viewing at least one video screen for abnormalities; c) sedation by a professional anesthetist; d) polyp search and/or removal during both insertion and withdrawal of the colonoscope; e) gradual insertion and withdrawal with a spiral or squared trajectory to maximize visualization of all mucosal surfaces including behind folds; f) tip retroflexion in the rectum; g) the utilization of copious amounts of irrigation to facilitate mucosal inspection even in cases with sub-optimal preparation; and, h) commitment to dedicate the resources to achieve complete exams (obtaining photo-documentation of the appendix in the cecum (i.e. “verification”)) in almost all cases.

Additional optional features of the methods, which are implemented prior to the colonoscopy, include specialized training of primary care physicians (“PCPs”), data mining of patient records and/or proper colon preparation.

Additional optional features of the methods, which are implemented after the colonoscopy, include quality assurance evaluation (optionally using a programmed computer system), patient and/or medical professional follow-up contact and/or reminders.

In an embodiment of the invention, PCPs are used as specialist-extenders, facilitating high-quality performance through appropriate technical and specialist backup support. A specialist-led, PCP-driven capacity expansion may be particularly relevant for reducing colorectal cancer disparities. African Americans, in particular, may benefit from such an expansion because of their unique risk profile (preponderance of, and rapid progress of proximal colon adenomas, earlier age of onset, and poorer outcomes at all cancer stages).

An aspect of some embodiments of the invention relates to a colonoscopy system configured to be used simultaneously by two or more attending medical professionals. In some exemplary embodiments of the invention, controls are provided to each of two attending medical professionals, wherein each set of controls is responsible for controlling different aspects of operation of the colonoscope of the system. For example, where one set of controls controls navigating (advancing, retracting, torqueing and/or maneuvering) of the colonoscope, where the second set of controls controls the tip of the colonoscope for performing medical procedures and/or visualizing the internal surface of the colon. In an embodiment of the invention, a controller is provided to the system which is programmed to account for the fact that two separate operators are simultaneously using the system.

There is provided in accordance with an exemplary embodiment of the invention, a method for improving the prevention of colorectal cancer in a patient, comprising: conducting a colonoscopy which includes a first medical professional navigating a colonoscope in a colon of the patient and, simultaneously, a second medical professional controlling a tip of the colonoscope to perform at least one medical procedure.

In an embodiment of the invention, navigating includes at least one of advancing, retracting, torqueing and maneuvering the colonoscope.

In an embodiment of the invention, a medical procedure includes at least one of identifying, sampling, destroying, removing and marking portions of the colon.

In an embodiment of the invention, the method further comprises administering anesthesia to the patient by a professional anesthetist, the third attending medical professional.

In an embodiment of the invention, the method further comprises performing additional actions to facilitate the colonoscopy, including at least one of patient repositioning, carbon dioxide gas insufflation, placing the patient in a Trendelenburg or reverse Trendelenburg position, spraying surfaces with vital dyes, applying abdominal external pressure, and warm water installation.

In an embodiment of the invention, the method further comprises training a primary care provider to perform colonoscopies using didactic instruction, use of models and simulators, and hands-on assistance by an expert.

In an embodiment of the invention, the method further comprises data mining including reviewing patient charts for upcoming patient visits to determine if the patient is up-to-date for colonoscopy, and if not to provide the patient with written and verbal advice to be screened.

In an embodiment of the invention, the method further comprises improving colon preparation by performing at least one of: removing patients from a colonoscopy schedule if they did not receive a preparation package; providing a note itemizing all bowel preparation instructions along with explanations; reminding the patient prior to the procedure to comply with colon preparation instructions.

In an embodiment of the invention, the method further comprises providing the patient with follow-up instructions at least partially based on the results of the colonoscopy.

In an embodiment of the invention, the method further comprises performing quality assurance using a specially programmed computer adapted to perform at least one of the following: measure fecal material removed during the colonoscopy; and, measuring percentage of mucosa inspected during the colonoscopy.

In an embodiment of the invention, the method further comprises documenting the colonoscopy using an attending medical professional. Optionally, alternatively and/or additionally, the documenting medical professional is the fourth medical professional participating in the procedure.

In an embodiment of the invention, colon visualization is performed during insertion and retraction of the colonoscope.

There is further provided in accordance with an exemplary embodiment of the invention, a method of performing colonoscopy using a team approach, comprising: navigating at least a portion of a colonoscope in the patient by a first attending medical professional; controlling at least a tip of the colonoscope to perform medical procedures by a second attending medical professional, wherein the controlling is simultaneous to the navigating of the first attending medical professional; providing anesthesia to a patient using a professional anesthetist, the third attending medical professional; and, documenting the location of discovered anomalies during the colonoscopy by a fourth attending medical professional.

In an embodiment of the invention, the fourth medical professional additionally creates at least one of a digital photo journal and a digital video journal.

In an embodiment of the invention, the fourth medical professional additionally performs all actions conducted through a biopsy channel during the colonoscopy.

In an embodiment of the invention, the method further comprises observing at least one video display of the colonoscopy by the first and second attending medical professionals, and at least one of: the third and fourth attending medical professionals.

In an embodiment of the invention, at least one of the attending medical professionals is at a remote location from where the colonoscopy is being performed.

In an embodiment of the invention, colon visualization is performed during insertion and retraction of the colonoscope.

There is further provided in accordance with an exemplary embodiment of the invention, a colonoscope for use by a colonoscopy performing team, comprising: controls configured for simultaneous use by two attending medical professionals, wherein the respective controls for each attending medical professional are insufficient, alone, to control the colonoscope to perform a colonoscopy.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example, are not necessarily to scale, and for purposes of illustrative discussion of embodiments of the invention. In this regard, the description taken with the drawings makes apparent to those skilled in the art how embodiments of the invention may be practiced.

FIG. 1 is a flowchart of a method for improving the prevention of colorectal cancer, in accordance with an exemplary embodiment of the invention;

FIG. 2 is a block diagram of a system for improving the prevention of colorectal cancer, in accordance with an exemplary embodiment of the invention;

FIG. 3 is a schematic showing the interrelationships between personnel, patient and system, in accordance with an exemplary embodiment of the invention;

FIG. 4 is a study cohort selection flowchart, in accordance with an exemplary embodiment of the invention;

FIG. 5 is a table of baseline characteristics of the study cohort at first colonoscopy, in accordance with an exemplary embodiment of the invention;

FIG. 6 is a table of Standardized Incidence Ratios of colorectal cancer incidence in the study cohort (relative to the South Carolina general population rate and national SEER-17 rates, in accordance with an exemplary embodiment of the invention; and,

FIG. 7 is a table showing the characteristics of 20 patients with incident colorectal cancer following initial colonoscopy, in accordance with an exemplary embodiment of the invention.

### DESCRIPTION OF SPECIFIC EMBODIMENTS OF THE INVENTION

The present invention, in some embodiments thereof, relates to cancer prevention and, more particularly, but not exclusively, to methods and systems for improving colorectal cancer prevention.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

The application includes sections which are arranged for providing clarity and organization. The sections and section headings should not be construed as necessarily limiting.

Generally

The inventor has made a number of observations and/or discoveries which have led to the development of the inventive methods and/or systems described herein. First, colonoscopy is a procedure that, in our current medical system, would usually be performed by a specialist that the patient has never met before. This intimidates many patients. Seeing an unknown medical professional in an unfamiliar place, planning to perform an uncomfortable and “invasive” test, makes them understandingly reluctant to proceed. Second, the challenge of distance also plays a factor for many among these non-doers because they live in more rural areas and have to travel to the closest metro area to have the procedure done—intimidating travel into unfamiliar locations, especially for older people. Third, this is accentuated among some patients that are hesitant to leave the familiarity and “comfort” of their personal PCP to be attended by gastroenterology specialists that feel different, less comforting, and even aloof, yet are about to perform a very invasive and intimate procedure. Fourth, there is an intense, perception-based fear of pain. This fear is accentuated with the concern of personal privacy, and the concern with what they might say or do under a sedated state. Fifth, even for those that can overcome some of these barriers and that seek the highest standard of care, there is little in the way of assure or information about quality and associated protective benefits (Adenoma Detection Rates (ADR) rates or other indicators) that might help them feel confident in their standard of care options.

Even if these five factors were not sufficient deterrents for procuring colonoscopies, the exorbitant cost (average nearly $2,000 under current policies) both direct (fees) and indirect (need for time off for pre-operative visits, procedure, post-operative visits) is beyond the reach many Americans. Even for those with insurance coverage, the out of pocket expenses (co-payment, deductible and co-insurance) average over $500. All these lead to procrastination and avoidance—decisions which in turn will result in a diagnosis of cancer for 3.9 million U.S. residents over the age of 50 sometime in their remaining lifetime.

In addition to the issues on the patient side, a maximally effective colonoscopy requires both technical expertise and a thorough inspection of the colonic mucosa, which calls for patience and skill on the part of the attending medical professional(s) to navigate through the folds and twists of the colon. Quality concerns on the physician side, including thoroughness and consistency, continually present issues related to colonoscopy procedures.

Exemplary Methods and Systems

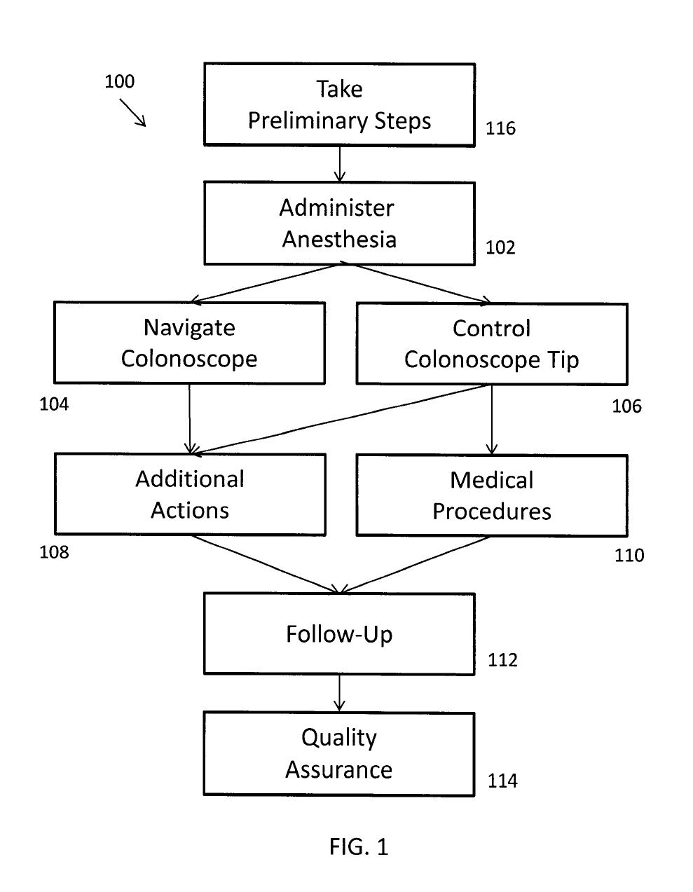

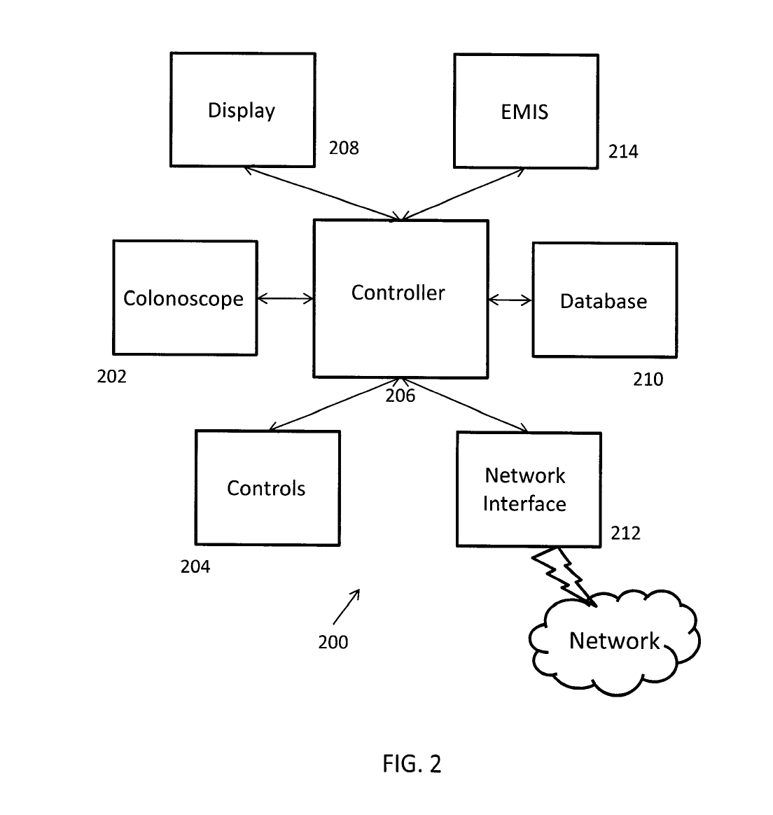

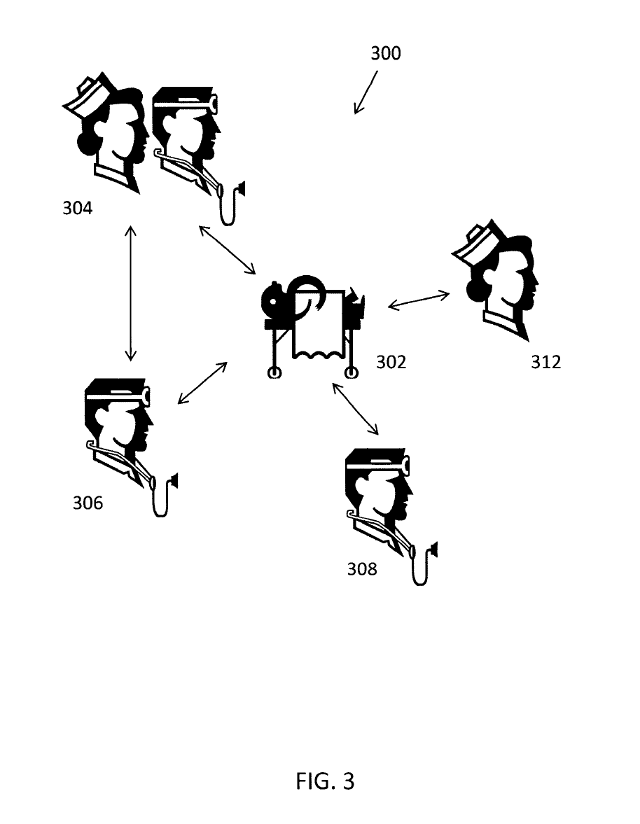

Referring now to the drawings, FIG. 1 illustrates a method 100 for improving and/or enhancing the prevention of colorectal cancer, in accordance with an exemplary embodiment of the invention. In some embodiments of the invention, features of the method 100 include: a) two-person colonoscope control technique; b) at least three or four persons viewing at least one video screen for abnormalities; c) sedation by a professional anesthetist; d) polyp search and/or removal during both insertion and withdrawal of the colonoscope; e) gradual insertion and withdrawal with a spiral or squared trajectory to maximize visualization of all mucosal surfaces including behind folds; f) tip retroflexion in the rectum; g) the utilization of copious amounts of irrigation to facilitate mucosal inspection even in cases with sub-optimal preparation; and, h) commitment to dedicate the resources to achieve complete exams (obtaining photo-documentation of the appendix in the cecum (i.e. “verification”)) in almost all cases. For ease of understanding of the method 100 of FIG. 1, FIGS. 2 and 3 are described next to introduce personnel and hardware which will be used in the detailed FIG. 1 desription.

FIG. 2 is a block diagram of a system 200 for improving the prevention of colorectal cancer, in accordance with an exemplary embodiment of the invention. In an embodiment of the invention, a colonoscope 202 is adapted for primary and/or simultaneous use by a plurality of attending medical professionals. For example, a first medical professional for navigating (including advancing, retracting, torqueing and/or maneuvering) at least a portion of the scope, while a second medical professional simultaneously controls at least a portion of the scope for visualization and treatment purposes. In some embodiments of the invention, the colonoscope is adapted by providing controls 204 which are operable by the same hand, of two different people (i.e. two different “right-hand” or “left-hand” controls are provided for use by two different people who are performing different simultaneous tasks). In some embodiments of the invention, a controller 206 of the system 200 is programmed to account for the fact that there are two different people operating the machine system simultaneously. For example, visual prompts are presented to each user depending on which tasks or actions the user is performing in the use of the system 200.

In some embodiments of the invention, at least one display 208 is provided to allow any attending medical professionals to visually observe the procedure. Optionally, each medical professional has a display 208 to enhance visual detection of anomalies in the colon. In some embodiment of the invention, the system 200 includes or is interfaced with a database 210, for example an electronic medical records database and/or a database for storing information and/or data related to the colonoscopy and/or patient. In some embodiments of the invention, the system 200 is networked using a communications interface 212, for example with a PCP's office, a remote database, a payer, the Internet and/or a medical professional who is participating (observing and/or actually performing actions) in the colonoscopy remotely.

In an embodiment of the invention, a quality-control focused, computer-controlled sub-system 214 is also provided to the system 200. For example, the Endoscopic Multimedia Information System (“EMIS”) developed by Jung Hwan Oh, Wallapak Tavanapong, Johnny Wong and Piet C. de Groen in conjunction with the University of North Texas, Iowa State University, the Mayo Clinic and Endometric, Inc. The EMIS is described in more detail in the Quality Assurance section, below.

FIG. 3 is a schematic 300 showing the interrelationships between personnel, patient 302 and system 200, in accordance with an exemplary embodiment of the invention. In an embodiment of the invention, at least two medical professionals simultaneously perform the colonoscopy using at least portions of the method 100. In some embodiments of the invention, at least one medical professional is a PCP 304. optionally, at least one medical professional is an expert 306 (such as described elsewhere herein). In some embodiment of the invention, at least one medical professional is not a physician, for example is a staff member 308 performing the role of the first attending medical professional (described in more detail below with respect to FIG. 1) and/or acting as a documentor/biopsy assistant 312 and/or providing another set of eyes for anomaly observation. In some embodiments of the invention, a professional anesthetist 310 participates in the colonoscopy procedure.

In an exemplary method for enhancing or improving the prevention of CRC, one, some and/or all of the following preliminary steps are taken (116), these steps occurring before an actual colonoscopy is performed, in accordance with an exemplary embodiment of the invention.
1. Training of PCP's—PCPs are trained to conduct colonoscopies for their patients in a highly structured and technically supported practice setting. Optionally, PCPs 304 are assisted by an expert level attending medical professional 306. The training process, post-training clinical performance protocol, and technical support mechanisms are designed to compensate for PCPs' lack of formal gastroenterology training, in an exemplary embodiment of the invention. In an embodiment of the invention, the training protocol consists of didactic instruction, use of models and simulators, and hands-on assistance by an expert 306, optionally up to the ASGE specified number of procedures for hospital credentialing (since August 2007, 140 procedures). The “expert” 306 is optionally at least one of a gastroenterologist, colorectal surgeon, or the director of a medical center's colonoscopy training program. The director can be a board-certified internist, trained and/or credentialed for colonoscopy and/or with hospital privileges. Over the course of training the PCP 304, hands-on assistance is gradually reduced to verbal assistance, sporadic manual assistance to navigate difficult turns. The expert 306 is optionally available for therapeutic assistance, for example to remove complex large, flat or cecal polyps. Training is described in more detail below in the Training Methods section.
2. Data Mining of PCP patient records—An optional component of the model is the improvement in compliance within the panel of patients of each PCP 304. A goal of the methodology is to improve uptake within practices by virtue of removing many of the barriers associated with the cost, inconvenience, fear and lack of confidence in an alternative provider for the service. In an embodiment of the invention, in-office staff is trained on “navigating” patients towards screening and/or to make financial concessions to hardship cases. The staff members are encouraged to review charts for upcoming patient visits to determine if the patient is up-to-date for colonoscopy, and if not to provide the patient with written and verbal advice to be screened, in some embodiments of the invention.
3. Colon Preparation—The standard bowel preparation regimen is prescribed (currently, 4 Dulcolax® tablets and 2 doses of 10 oz. citrate of magnesia, 4 hours apart, with additional dietary and fluid intake guidelines). In an embodiment of the invention, to maximize ensuring good bowel preparation, no patient 302 will remain on the schedule that did not receive the preparation package from the provider (or directly from the facility where the colonoscopy will be performed). Each patient 302 receives a note itemizing all the bowel preparation instructions, along with item-wise explanation by the PCP 304. These instructions are reinforced by a staff member, for example a nurse, calling the patient prior to the procedure. For example, 3 days prior to the procedure. It has been shown by the inventor that this focus on adequate preparation has had a dramatic impact on reducing cases cancelled due to inadequate bowel preparation. In national studies, often fifteen percent of cases are incomplete, often due to poor laxative preparation. In an embodiment of the invention, this is reduced to less than one percentage point.

In a procedure performed millions of times annually, the additional expense of an anesthesia provider is a relevant issue. For this reason, the physician performing the colonoscopy is also responsible for delivering the “conscious sedation” for the patient in the traditional colonoscopy model. Whereas this may confer a cost savings, it has been proven to increase the danger of sedation related complications (sometimes fatal) due to the doctor focusing on the case, rather than the patient's vital signs. It has also been clearly demonstrated that the quality of the exam is enhanced with adequate anesthesia. In the traditional colonoscopy, the intravenous sedation is administered and the physician introduces the scope and attempts to achieve the maximal insertion (to the cecum) rapidly while the medication remains effective. The scope withdrawal phase is associated with minimal discomfort, so additional sedation is seldom necessary during this phase. On withdrawal, occasionally the scope “falls out” of a sharp turn. If the patient is no longer adequately sedated, the physician may have a subconscious dis-incentive to retrace the region due to an adverse patient response.

With “monitored anesthesia care” by professional anesthetists, the patient remains asleep throughout the test so that the attending medical professional(s) can focus one hundred percent of their attention on the task at hand: prevention of colorectal cancer. In an embodiment of the invention, anesthesia is administered (102) by a professional anesthetist 308. Another advantage of using a professional anesthetist 308 is that the PCPs 304 are un-accustomed to cardio-pulmonary resuscitation, which is sometimes necessary. In an embodiment of the invention, the sedation protocol consists of propofol slow intravenous administration titrated to patient response.

The initial colonoscopes were fiber optic devices that limited the internal visualization of the lumen being investigated to the operator of the fiber optic colonoscope. With this initial technology, no one else could directly visualize the status of the exam, so assisting with scope manipulation (advancing, withdrawing, torqueing) was potentially dangerous. With the advent of video technology, the fiber optic bundle has been replaced with a three-chip charge coupled device camera chip. Gastroenterologists are often instructed by academic Gastroenterologists who were taught in the pre-video era. Subsequently, the prevailing wisdom for colonoscopy is that the existing techniques are not in need of improvement. Colonoscopy takes years to learn, and by the time the basic functions are successfully mastered, the academic journey is at an end. Gastroenterologists at the completion of training often are still functioning below the industry standards in terms of quality metrics (cecal intubation, polyp detection, complication rates, ADR).

The earlier generation colonoscopes are stiffer than the current generation. Many physicians prefer the more flexible scopes and often employ the “pediatric” versions, even for adult cases. It is possible to break down colonoscopists into the two ends of a spectrum which might be characterized as the “damn the torpedoes” versus the “delicate finesse”. Surprisingly, each has its advocates and each can be effective. With the former, the scope is advanced through the tortuous colon using the distal bending tip to find additional lumen as the scope is advanced towards the cecum. In the second, the scope is rotated delicately so that the colon is gradually straightened out so that the advancement is made easier. The colon is usually unattached within the abdomen and the colonoscope can act as a conduit that permits the colon to be gradually telescoped onto the shaft. Advancing the scope, withdrawing the scope, and twisting the scope all facilitate the ability for the physician to reach the cecum with minimal looping or twisting of the colon.

In an embodiment of the invention, the method 100 employs a team approach for maneuvering the colonoscope 202 and performing the colonoscopy. For example, in an embodiment of the invention, a first attending medical professional advances, retracts and/or maneuvers (104) the scope while a second attending medical professional controls (106) the deflection of a tip of the scope 202, while optionally, also visualizing and excising polyps. Optionally and/or alternatively, an additional person or persons are used for performing some or all of the advancing, retracting, maneuvering, controlling, visualizing and/or excising. Optionally, at least one of the actions described above is performed by a different person than described, for example the second attending medical professional maneuvers. In addition, the method optionally includes (108) additional actions, for example at least one of patient repositioning, carbon dioxide gas insufflation, gravity (Trendelenburg or reverse Trendelenburg), spraying surfaces with vital dyes (indigo carmine), abdominal external pressure and/or warm water installation, one, some and/or all of which are used to assist navigation of the colon and/or visualization of the internal surface of it.

In an embodiment of the invention, this methodology 100 permits visualization of a large portion of the mucosal surface during the insertion phase, the nearly straight colon anatomy provides an optimal anatomic configuration for maximal visualization of the colon surfaces upon withdrawal. In addition to allowing for less tortuous navigating of the colon, the two-man technique provides an additional set of eyes to identify polyps.

In some embodiments of the invention, a fourth person 308 (the third being the anesthesiologist 310) is added into the colonoscopy procedure: a documenter/biopsy assistant. This protocol not only provides a fourth set of eyes, but also has demonstrated an increased occurrence of finding polyps as well as a reduction of complications (from over-cauterization). In some embodiments, an on-site expert is provided for rescue assistance (either navigational or therapeutic).

In an embodiment of the invention, the use of two attending medical professionals to perform advancing, retracting and/or maneuvering (104) the scope and, separately, the controlling (106) provides benefits including: (a) the dexterity of 2 “right-hands,” (b) having the skill of a second technically trained person to provide operational and clinical support; and, (c) preempting or forestalling motor skill deterioration due to forearm fatigue.

In an embodiment of the invention, the first attending medical professional is a procedural technician, not necessarily a physician. Optionally, the first attending medical professional is a physician. In some embodiments of the invention, the second attending medical professional is a physician, for example a PCP 304. Optionally, a physician is also an “expert” 306, as described elsewhere herein.

In an embodiment of the invention, PCPs 304 perform their cases at an ambulatory surgical center (“ASC”) after training (optionally, with an experienced gastrointestinal/procedural technician assisting with scope manipulation), and/or optionally, an on-site expert 306 available for backup assistance. In some embodiments of the invention, the on-site expert is frequently engaged in training other PCPs or performing their own procedures, but remains on-call as an on-site backup. The expert also optionally performs quality assurance oversight duties, such as described in more detail below in the Quality Assurance section.

In an embodiment of the invention, colonoscopies are conducted using a video-colonoscope 202 with variable stiffness (for example, the Olympus® America video-colonoscope 160 series, predominantly PCF 160 AL), with the first attending medical professional advancing, withdrawing and manipulating (104) the scope (does not have to be a physician) and the second attending medical professional controlling (106) the scope. In an embodiment of the invention, during initial training, the first attending medical professional is a physician, and then gradually evolves towards a procedure tech. As described elsewhere, at least two people perform the method 100, but optionally three, four or more participate, for example at least one physician, an anesthetist, a procedure tech and/or a documentor/biopsy tech, some or all of which observe the video display(s) 208 to assist with polyp detection, thereby minimizing missing of lesions/polyps due to concentration lapses, visual error and/or visual/motor fatigue.

In some embodiments of the invention, residual stool is flushed during the colonoscopy to ensure maximally effective examination for polyps.

An objective of the method 100 is the successful attainment of the end of the colon (i.e. the cecum) and verification that the cecum has been reached. In an embodiment of the invention, during retraction (104), the tip of the scope is controlled (106) to visualize as much of the interior colon surface as possible. This is optionally achieved by slowly withdrawing (104) the scope and controlling (106) the tip of the scope 202 to move in an extended circular or square pattern in an effort to observe all interior surfaces of the colon. The motion of the bending section flattens the natural folds facilitating visualization of previously hidden regions. In some embodiments of the invention, observing the interior of the colon is also performed during insertion/advancing.

Either during scope withdrawal or during advancement, or during both, at least one of the attending medical professionals performs (110) at least one medical procedure, including: identifying polyps, sampling tissue/polyps, destroying tissue/polyps, removing tissue/polyps and/or marking the colon. As described herein, in some embodiments of the invention, the medical professional performing controlling (106) identifies, samples, destroys, removes and/or marks the colon.

Patients with significant adenomas were advised (112) to return for surveillance colonoscopy 1-5 years later (depending on adenoma features), and the remaining, after 10 years. Compliance is at the patient's discretion but is supported by a recall database allowing for telephone call(s), letters to referring physicians and letters to the patient to provide follow-up information and/or reminders.

In an embodiment of the invention, quality control metrics are employed (114), such as described in more detail below in the Quality Assurance section.

Quality Assurance

A high-quality colonoscopy requires both technical expertise and a thorough inspection of the colonic mucosa, which calls for patience and skill to navigate through the folds and twists of the colon. Because of the strong link between performance quality and the CRC protective effect of screening colonoscopies, much research has been performed to assist in identification of the factors contributing to high levels of protection. Professional organizations have evaluated this research and postulated guidelines for evaluation of the quality of colonoscopy screenings. Foremost among these are the American College of Gastroenterology (ACG), the professional organization that confers upon Gastroenterologists that esteemed status of board certification. A second premier entity is the American Society for Gastrointestinal Endoscopy (ASGE), which in addition to Gastroenterologists, includes other physicians performing colonoscopy. Together, these two organizations developed the ASGE/ACG Taskforce on Quality in Endoscopy which has established quality indicator norms for colonoscopy, including cecal intubation rates (reaching the end of the large intestine) (95%), withdrawal time (minimum=6 minutes), adenoma detection rates (minimum of 25% in men and 15% in women), and colon perforation rates below (0.1%). In addition to these quality indicator norms, the Taskforce also recommended that mucosally based pedunculated polyps and sessile polyps less than 2 cm in size should not be sent for surgical resection without an attempt at endoscopic resection or documentation of endoscopic inaccessibility.

Cecal intubation, while a positive event for each colonoscopy, merely provides evidence of the fact that the entire colon was traversed. In terms of ADR, a physician could find one polyp and meet the criterion. However, the discovery of one or more polyps is not the same as complete removal of all polyps in the colon. Similarly, a six-minute withdrawal time does not guarantee that all mucosa was cleaned as needed and also does not provide a measure of the percentage of the mucosa inspected. In summary, the current colonoscopy minimal quality criteria do not provide a complete picture of true colonoscopy quality. While they do indicate positive activities and events that should occur during a colonoscopy, they do not indicate the state of colon preparation, the percentage of mucosa inspection, or the completeness of polyp removal. The current limited data provide a false sense of measuring or providing quality and allow for easy manipulation by the physician (e.g. a physician could remove one polyp and remove the endoscope slowly without looking around the mucosa or within the folds to merely reach the six minute mark) to skew the procedure data toward an apparently favorable outcome.

While these indicators/recommendations do reflect positive activities and events that should occur during a colonoscopy, they fail to reflect important procedure outcomes that have a significant impact on the quality of the procedure. Important procedure outcomes which should be considered include: the extent of colon preparation involving thorough removal of remaining fecal material (Colon Preparation); the percentage of mucosa inspected (% inspected); and, the completeness of lesion removal (Complete Polyp Removal).

The inventor believes that PCPs' high performance on cecal intubation (and lesion detection) is attributable to a combination of factors: a uniformly applied protocol of bowel preparation that is regularly updated, a standard protocol for ensuring that patients understand and are provided timely instruction reminders, and/or PCPs performing in a highly structured and supported environment designed to compensate for their lack of formal gastroenterology training. A careful and exhaustive mucosal inspection, and high lesion yield rates are also facilitated by an emphasis on a team approach, where all persons present, including the PCP, the assisting technician, the nurse anesthetist, and/or the documenter observe the video-screen, thus increasing the polyp detection rate and ensuring cecal intubation. Finally, if the expert determined that a case is high-risk, the PCP will not perform the procedure. Exemplary conditions that trigger patient referral to the expert are frail or elderly patients aged over 75 years, and/or presence of acute symptoms or medically unstable conditions.

In an embodiment of the invention, cecal intubation or satisfactory completion is defined based on either, or both, of the following criteria, as appropriate: (a) visualizing the appendiceal orifice, and when not seen, visualizing both cusps of the ileocecal valve or crow foot in the cecum (photographs documented in all cases); and, (b) intubating the ileal tip and identifying lymphoid follicles to verify that complete navigation of the colon was achieved.

The method 100 emphasizes a careful and gradual polyp search and removal during both insertion and withdrawal, because polyps found during insertion are sometimes not re-identified during scope withdrawal. Since it is well known that approximately 20% of polyps are missed even by the most accomplished colonoscopist, it is inadvisable to assume that small polyps will be re-identified during the withdrawal phase. Therefore for quality assurance, in an embodiment of the invention, documentation of both insertion and withdrawal times is performed.

In some embodiments of the invention, other measurable quality indicators are tracked, including: overall cecal intubation rate; the US Multi-Society Task Force criterion-adjusted intubation rate (excluding incomplete cases because of severe colitis and poor preparation); circumstance-adjusted cecal intubation rate (excluding incomplete cases because of poor preparation, tortuous colon, obstruction or stricture, severe colitis or diverticulosis, vital sign instability, prior surgical removal of the cecum, and patient discomfort); percent of patients with lesions classified by a combination of polyp morphology and histology, for example any polyp (growth with normal histology, hyperplastic, adenomatous or cancerous histology), adenomas (adenomatous histology with tubular, serrated, villous or tubulovillous features, or cancer), hyperplastic polyps, advanced neoplasms (adenomas 10 mm in diameter, villous histologic features, high grade dysplasia or cancers), cancer, and/or carcinoids; mean adenomas per case; and/or, mean advanced neoplasms per case.

In an embodiment of the invention, quality assurance is supplemented by the use of a quality-control focused, computer-controlled system, for example the EMIS, described above. In an embodiment of the invention, the EMIS is programmed to analyze a digitized video file of the colonoscopy procedure and/or produce metrics that are designed to reflect the quality of the procedure. These quality metrics can be used to evaluate and improve the physician's skill and colonoscopy procedure quality. In an embodiment of the invention, the final quality of a colonoscopy is comprised of at least one of several components that are patient-related (e.g. colon preparation), equipment-related (e.g. quality, color, and contrast of the image, automatic light adjustment characteristics of the light source), and physician-related (e.g. maximal extent of insertion, withdrawal time, effort of inspection of the visible mucosa, instrumentation technique, appropriate selection of instruments for specific lesions). Ultimately, the full measure of colonoscopy quality is comprised of several different quality measures.

The EMIS is programmed, in an embodiment of the invention, to utilize the entire procedure to determine quality. This is in contrast to merely taking a few convenient, easily measurable, multiple, procedure-based features, such as cecal intubation rate, adenoma detection rate, and average withdrawal time, such as is done currently. Instead of providing an average, subjective, surrogate rating of quality for a physician, that is meaningless for an individual patient, the EMIS provides an objective, detailed report of an individual procedure reflecting the actual quality provided to an individual patient, in an embodiment of the invention. The solution is algorithm-based, automated analysis of the video stream representing the entire procedure. In some embodiments of the invention, the EMIS is programmed to provide an objective measurement of at least one of three important aspects of colonoscopy quality: Colon Preparation, Percentage of Mucosa Inspected, and Complete Polyp Removal.
1. Colon Preparation—the patient should adhere to the colon preparation instructions (which is reinforced by the method 100, which includes the preliminary steps described above) and the attending medical professional optionally removing any remaining fecal material (which can be measured by the EMIS).
2. Percentage of Mucosa Inspected—in an embodiment of the invention, the physician looks behind every fold (working the folds) and/or moves or removes remaining stool to achieve as close to 100% inspection of the colon mucosa as reasonably possible, and which can be objectively measured by the EMIS. The EMIS programmed to measure the percentage of mucosa inspected is a new breakthrough, which will be the first objective measurement of how much of the colon was actually seen by the physician.
3. Complete Polyp Removal—in an embodiment of the invention, the physician removes all polyps by biopsy forceps, snare polypectomy, or other modalities, since lesions left behind may develop into a malignant lesion before a next screening or surveillance procedure is performed. The method 100, in combination with the EMIS, ensures greater polyp recognition, particularly since the EMIS is optionally programmed to provide visual cues to indicate potential polyps.

It is believed by the inventor that the method 100 (including preliminary and supplemental procedures, as described herein) in combination with the EMIS, which is specifically programmed to complement the method 100, will lead to significant improvements in colonoscopy quality that can be fully documented and objectively measured. The combination of a rigorous clinical protocol with the ability to objectively measure the quality of the protocol will equip all physicians with a tool that will lead to a world class CRC prevention tool.

Teaching Methods

In an embodiment of the invention, the teaching/training protocol consists of at least one of three major components: didactic instruction; use of models and simulators; and, hands-on assistance from an expert up until a PCP reaches the ASGE specified number of procedures for hospital credentialing.

In an embodiment of the invention, the training methods described herein and the intraprocedural protocol developed by the inventor are combined with the intent to meet or exceed the ASGE and ACG criteria as well as address the additional criteria of the final state of colon preparation, the percentage of mucosa inspected, and the completeness of lesions removal. Further, this training method and intraprocedural protocol are optionally combined with a specially programmed EMIS to provide an objective quality measure of each colonoscopy. The goal of combining the teaching protocol with the EMIS is to produce a training program with real-time feedback and quality measurement that can equip physicians to perform high quality colonoscopies.

In an embodiment of the invention, procedures are overseen by a gastroenterologist, a colorectal surgeon or the director of the ambulatory surgical center. After several procedures, the expert moves from hands-on assistance to verbal assistance with sporadic intervention when difficult turns must be navigated or irregular polyps must be removed. In an embodiment of the invention, all procedures performed by PCPs require two people. As described above, procedures are optionally performed by two, three, four of more people, with the actual personnel used and/or the number of personnel used varying depending on the stage of training. For example, early in training the expert navigates the scope through the colon while the PCP searches for and removes polyps. Once training is complete, an experienced gastrointestinal technician assists the PCP, and optionally an expert is on site to address any complications.

Employing the above described inventive methods and systems, advantages over the traditional model include:

The rapid induction and recovery of Propofol improves turnover time.

The lack of patient retrograde amnesia with Propofol allows physician review of findings with patients and their family/companion to be done upon discharge, not at another appointment following the procedure.

Propofol anesthesia is clearly preferred by patients allowing them to resume a full remainder of the day activities unlike the variable sedative effects of traditional Demerol/Versed conscious sedation. (Studies at research centers are being conducted to explore the potential for patients to even drive themselves home. Traditional Demerol/Versed sedation absolutely impairs driving, but with the rapid clearance of the non-narcotic Propofol, protocols may be established to allow save vehicular usage after a colonoscopy.)

Dedicated patient anesthesia and intra-procedure monitoring by a professional anesthetist appears clearly superior to sedation delivered by the endoscopist or RN who are experienced with conscious sedation by virtue of education and procedural anesthesia/sedation studies. The constant presence of an anesthetist trained to prevent cardio/pulmonary emergencies by early detection and prompt treatment cannot be underestimated. And in the event of just such an emergency, the same level of anesthetist professional competence can handle it.

CO₂ (Carbon Dioxide) is used for all cases, decreasing the need for withdrawing gas from the colon on withdrawal as is done with air, (and thereby decreasing optimal visualization). Because the CO₂ is absorbed by the body ten times faster than air, it virtually eliminates patient gas discomfort after the procedure.

All procedural documentation is completed during the examination by a technician (the fourth attending medical professional) so paperwork requirements by the physician are almost eliminated with the exception of a confirmation by physician signature. This is not only a boon for prompt completion of the medical record, but also because it occurs “real time,” documentation details are not left to the memory of a physician who might have done a number of procedures before recording findings. This ensures timely, accurate documentation for patients.

A strict set of safety protocols for the instrumentation nurse (the fourth attending medical professional) prevents untoward complication for both patients and the expensive endoscopic equipment. Use of cautery devices is still done, (as required by law), by a licensed physician, however, the processes used mitigates individual human error and variation of operator skill levels by consistent use of “safety first” pathways.

The entire design of the team approach encourages situation awareness by all team members that creates a safer, higher quality, and more efficient colonoscopy experience.

Eight eyes are better than three eyes! For the physician endoscopist, these extra eyes are a powerful element of ensuring patients a high quality colonoscopy. Each member of the team is well versed on the components of a thorough examination and as a team, no one member of the team is tempted to take timesaving shortcuts resulting in a marginal colonoscopy quality.

The skill sets necessary for a PCP to master as part of the team approach can be acquired in a fraction of the time a solo endoscopist and generates superior results overall because of a redefined authentic team effort. The study by Dr. Sweeny demonstrated the three primary quality metrics, completion rate (getting to the cecum for a full colonoscopy), polyp yield (ADR), and complications (avoidance of cardio-pulmonary problems, colonic perforation, etc.) are actually all the lowest during a patient's first colonoscopy. The inventive methods described herein using the team approach improves all of these parameters.

Study of Clinical Results

Reference is now made to the following clinical study and results, which together with the above descriptions illustrate some embodiments of the invention in a non-limiting fashion.

The inventor performed an investigative study of current and former patients of the inventor's ambulatory surgical center on which methods and/or systems described herein (or variations thereof) were at least partially employed.

Patients

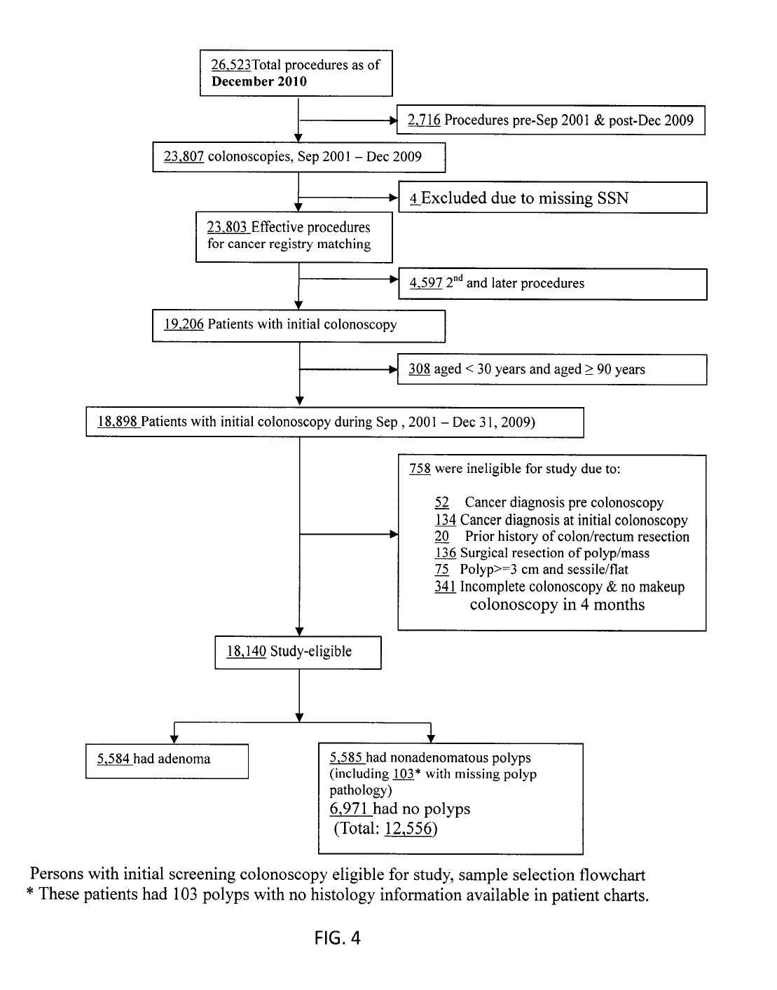

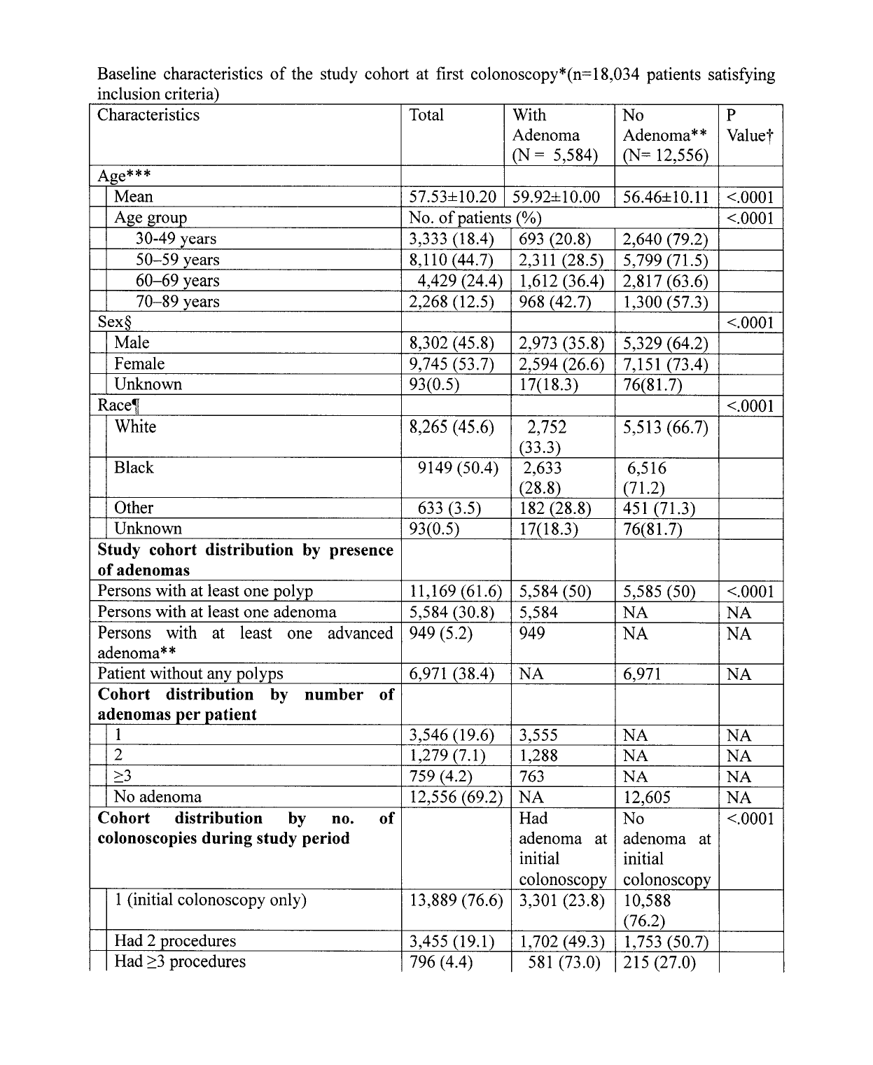

The study cohort consisted of over 18,000 persons with their first colonoscopy between Oct. 1, 2001 and Dec. 31, 2009, performed by 58 physicians. Polyps were removed at colonoscopy except for invasive cancers or complex polyps felt to be clinically beyond the capability of the screening team, and these cases were referred for surgical excision. FIG. 4 shows the study cohort selection flowchart as applied to the center's database of all colonoscopies conducted at the center as of Dec. 31, 2010.

Comparison Group

The South Carolina general population was the comparison group, used to generate the expected number of cases in the study cohort by applying the South Carolina population-wide age-, sex-, race- specific incidence rates for 2001-2008, to the study cohort's person-years of observation (PYO) stratified into age-, sex- race-strata. The standardized incidence ratio (SIR) was the observed divided by expected number of cases.

End Points

CRC incidence was the end point, determined by matching the study cohort with the South Carolina Central Cancer Registry (“SCCCR”) cancer database. The SCCCR epidemiologist conducted a manual review and populated the match field. The full list of all patients (match or no match) was subjected to repeat, independent manual review by the study team, followed by patient chart reviews for all poor matches that held any remote possibility of being a true match. The second manual review yielded one new cancer match to the original match list determined by the SCCCR epidemiologist (identified from among those with changed last name by matching on residential address). The censoring second end point was death of cancer patients (CRC or other cause noted), ascertained from the Vital Records Registry. The end points used to censor a patient's eligibility for inclusion in a given year's PYO pool were month/year of cancer diagnosis or death. All persons with either a CRC diagnosis or CRC death were defined as CRC incidence cases.

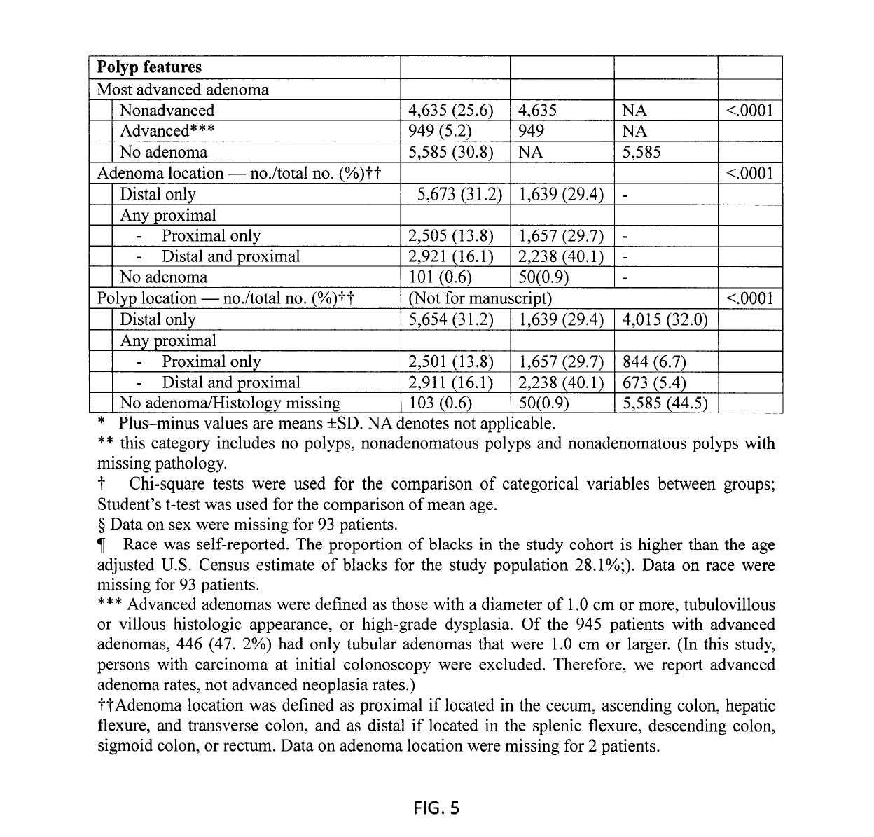

The distribution of the 18,140 study patients, 5,584 with adenoma(s) at initial colonoscopy and 12,556 without adenomas by demographic and initial colonoscopy characteristics are presented in FIG. 5, a table of baseline characteristics of the study cohort at first colonoscopy. The cohort was predominantly female, black, and had an adenoma rate of 30.8%.

All study eligible cases were subjected to analysis to calculate person years at risk and stratified into age-sex-race sub groups to calculate expected number of cases at the general population incidence rate.

Reporting on the performance of a novel PCP-delivered colonoscopy program, we found that the rates of cecal intubation and adenoma detection as well as mean endoscopy withdrawal time meet or exceed the ASGE benchmarks. Uniquely, the SCMEC's protocol requires colonic inspection for polyps during both intubation and scope withdrawal. Our observed cecal intubation rate (98.1%) is higher than the ASGE standard of 95% for screening colonoscopies, which is generally met by gastroenterologists. Compared with the documented (highly variable) rates of 49% to 95% for PCP-performed colonoscopies, our study rate is higher and more uniform across physicians. Variations in cecal intubation rates are due to physician variables (skill including dexterity, training level), patient variables (age, gender, body mass index, past surgeries, tortuous colon, pain threshold, and response to anesthesia), and adequacy of bowel preparation. Adequate bowel preparation facilitates higher cecal intubation rates. In our cohort, 91% of patients were documented to have excellent to fair bowel preparation, which is relatively high.

The study PCPs' high rates of adenoma detection in men (34.6%) and women (25.4%), exceeding the ASGE benchmarks (men 25%; women 15%), may be due to patient variables such as age, gender, and race. African Americans (AAs) constitute about half of this study population, and it is thought that adenoma rates are often higher in AAs. The major adverse event rate of 0.06% (that included 2 perforations, both perforated diverticulae, unrelated to scope advancement or polyp removal; no death) is statistically similar to the documented 0.067% perforation rate for specialist-performed screening colonoscopies. Two large-scale studies on screening procedures reported death rates of 0.074/ 1000 and 0. The dynamics of perforation due to barotrauma are documented, as are mechanical accidents during navigation and ileal intubation, and electrocautery/thermal injury mishaps. These injuries are linked primarily to suboptimal dexterity and technical judgment. Again, the study PCPs' low complication rate is plausibly because of the 2-person technique, specialist backup, and referral of potentially high-risk patients to the specialist for evaluation.

In-house evaluation of the frequency of expert rescue assistance among post-training procedures yielded a rate of 14.0% (manual or therapeutic assistance) among 406 consecutive post-training procedures performed by PCPs from November 2006 to February 2007.

A board-certified colorectal surgeon evaluated the photographic evidence of all 1112 PCP colonoscopies conducted from Jul. 1, 2006 to Jun. 30, 2007, which were recorded by the PCP as “cecal intubation completed” and 99.6% (1108 cases) of those cases were verified as accurate.

At the expected rate of CRC, the program could save $4 for every $1 spent on screening (assuming a cost of $1,000 per colonoscopy).

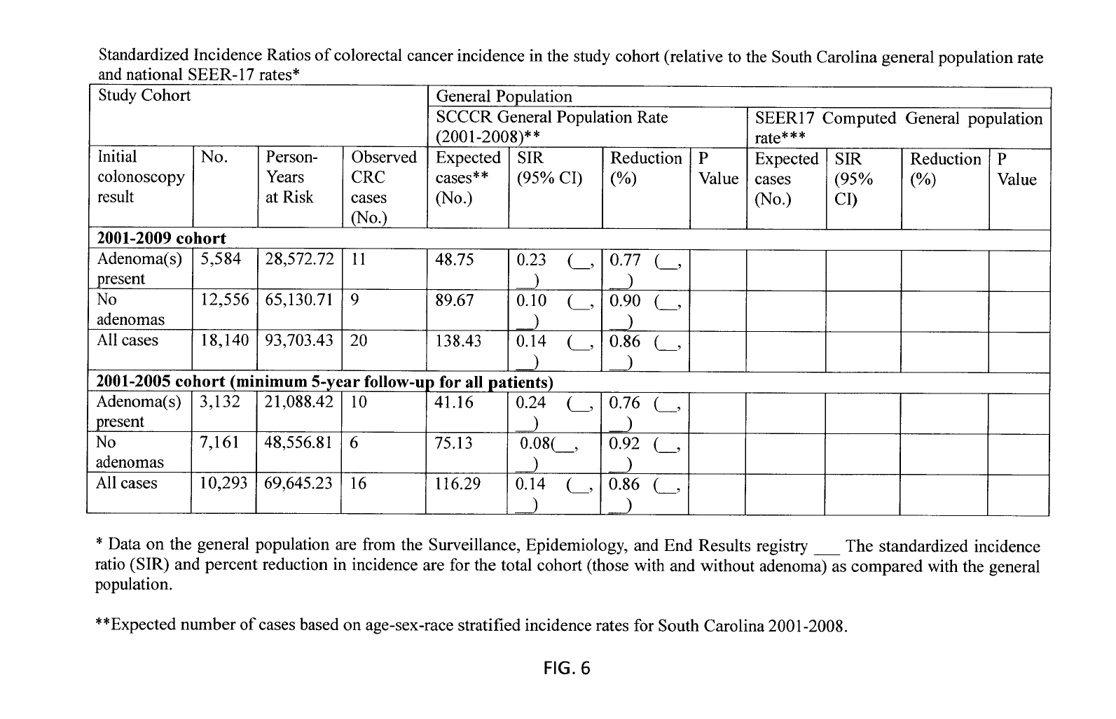

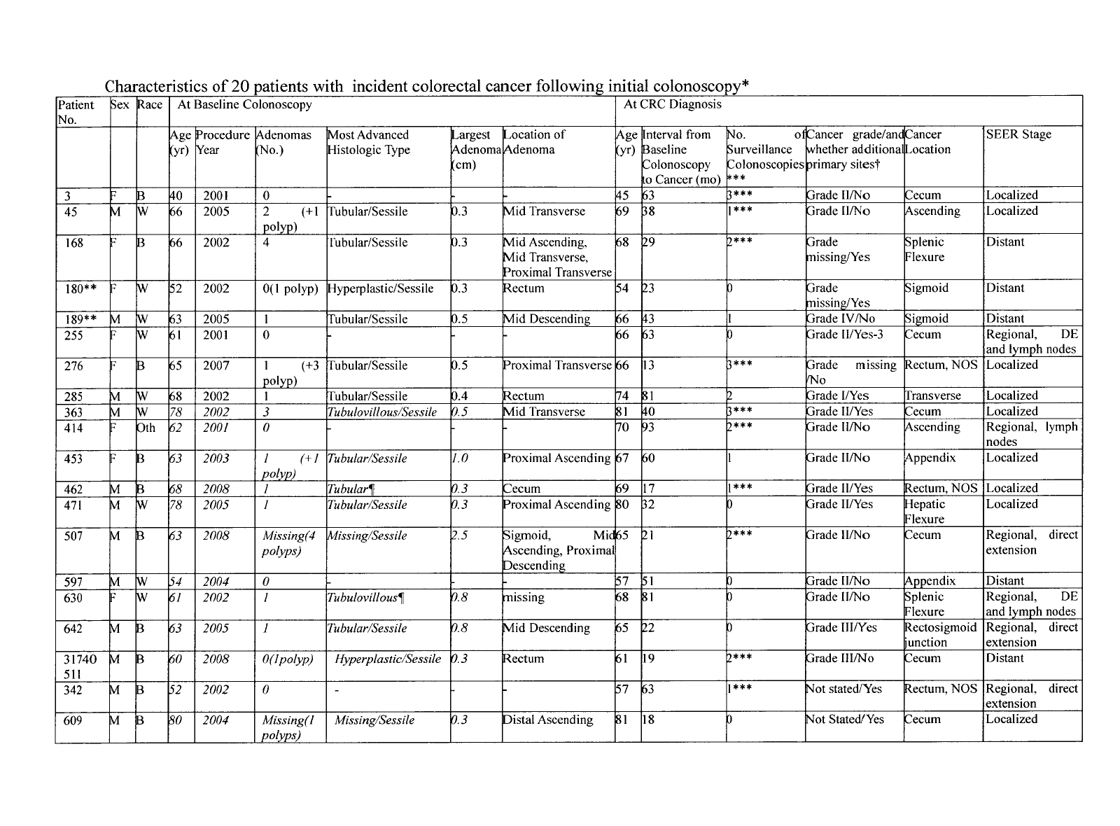

To evaluate the long term cancer protection rate of the center's colonoscopy series, we conducted additional analysis limiting the cohort to those with initial colonoscopy as of Dec. 31, 2005 to ensure at least 5-year follow-up for all cases. We present the SIRs (relative to the SCCCR population incidence rate, and the SEER-17 incidence rate) and exact 95% confidence intervals assuming the observed number of incidence cases to follow a Poisson distribution, using a two-sided p-value of 0.05 for statistical significance. For the observed incident cancer cases, we present demographics, polyp/adenoma status and most advanced histology at initial colonoscopy, colonoscopy-to-cancer interval, whether diagnosed at surveillance colonoscopy, presence, type and timing of additional primary cancers at other sites, SEER summary stage, and anatomic location. In our study, the protection rate among the 5-year follow-up subgroup was 95% (as shown in FIG. 6). The observed high cancer protection validates the inventive methods and/or systems described herein as being effective in reducing CRC, many recently documented as effective in increasing polyp detection rates.

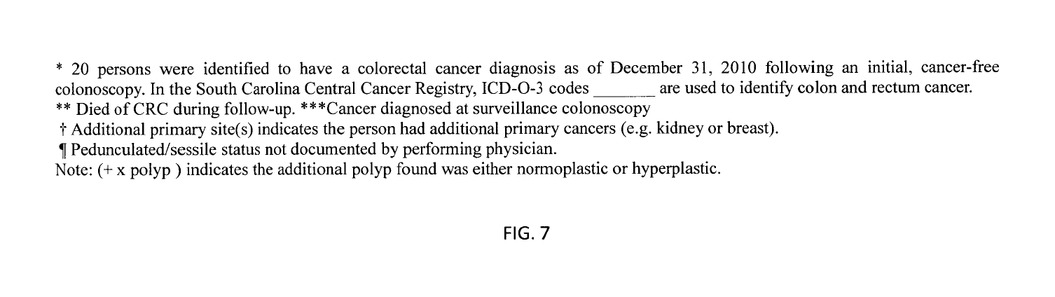

FIG. 7 is a table showing the characteristics of 20 patients with incident colorectal cancer following initial colonoscopy. Of the 20 colorectal cancer cases despite colonoscopy (CCdP) that were identified, 10 were black patients and 8 were female patients. These patients were diagnosed with CRC 13-93 months from initial colonoscopy, and 10 persons had additional primary cancers at a non-colorectal site (one patient had 3 additional primary cancers). Of 20 with colorectal cancer, 14 had one or more surveillance colonoscopies, and 11 were diagnosed at a surveillance colonoscopy. At initial colonoscopy 12 patients had had sessile polyps/adenomas removed, although none of the earlier polyp anatomic locations coincided with that of the cancer.

Our study suggests that colorectal cancer is in fact highly preventable by well-performed screening colonoscopies accomplished by the methods and/or systems described herein, completed by reaching the cecum, and under consistently enforced polyp-maximizing procedure protocol across all endoscopists.

Because all colonoscopies were routine practice cases reimbursed by the prevailing payer mix (without additional “research” funding) without jeopardizing the financial viability of the center, the study validates long-term sustainability of the clinical protocol. Protocol observance could be ensured across 58 physicians over 10 years, suggesting that clinically accountable care protocols can overcome resistance arising out of perceived threats to personal clinical autonomy.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, on-patent literature patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting, they are for clarity.

What is claimed is:

1. A method for improving the prevention of colorectal cancer in a patient, comprising:
conducting a colonoscopy which includes a first medical professional navigating a colonoscope in a colon of the patient and, simultaneously, a second medical professional controlling a tip of the colonoscope to perform at least one medical procedure.

2. A method according to claim 1, wherein navigating includes at least one of advancing, retracting, torqueing and maneuvering the colonoscope.

3. A method according to claim 1, wherein a medical procedure includes at least one of identifying, sampling, destroying, removing and marking portions of the colon.

4. A method according to claim 1, further comprising administering anesthesia to the patient by a professional anesthetist, the third attending medical professional.

5. A method according to claim 1, further comprising performing additional actions to facilitate the colonoscopy, including at least one of patient repositioning, carbon dioxide gas insufflation, placing the patient in a Trendelenburg or reverse Trendelenburg position, spraying surfaces with vital dyes, applying abdominal external pressure, and warm water installation.

6. A method according to claim 1, further comprising training a primary care provider to perform colonoscopies using didactic instruction, use of models and simulators, and hands-on assistance by an expert.

7. A method according to claim 1, further comprising data mining including reviewing patient charts for upcoming patient visits to determine if the patient is up-to-date for colonoscopy, and if not to provide the patient with written and verbal advice to be screened.

8. A method according to claim 1, further comprising improving colon preparation by performing at least one of: removing patients from a colonoscopy schedule if they did not receive a preparation package; providing a note itemizing all bowel preparation instructions along with explanations; reminding the patient prior to the procedure to comply with colon preparation instructions.

9. A method according to claim 1, further comprising providing the patient with follow-up instructions at least partially based on the results of the colonoscopy.

10. A method according to claim 1, further comprising performing quality assurance using a specially programmed computer adapted to perform at least one of the following: measure fecal material removed during the colonoscopy; and, measuring percentage of mucosa inspected during the colonoscopy.

11. A method according to claim 1, further comprising documenting the colonoscopy using an attending medical professional.

12. A method according to claim 11, wherein the documenting medical professional is the fourth medical professional participating in the procedure.

13. A method according to claim 1, wherein colon visualization is performed during insertion and retraction of the colonoscope.

14. A method of performing colonoscopy using a team approach, comprising:
navigating at least a portion of a colonoscope in the patient by a first attending medical professional;
controlling at least a tip of the colonoscope to perform medical procedures by a second attending medical professional, wherein the controlling is simultaneous to the navigating of the first attending medical professional;
providing anesthesia to a patient using a professional anesthetist, the third attending medical professional; and,
documenting the location of discovered anomalies during the colonoscopy by a fourth attending medical professional.

15. A method according to claim 14, wherein the fourth medical professional additionally creates at least one of a digital photo journal and a digital video journal.

16. A method according to claim 14, wherein the fourth medical professional additionally performs all actions conducted through a biopsy channel during the colonoscopy.

17. A method according to claim 14, further comprising observing at least one video display of the colonoscopy by the first and second attending medical professionals, and at least one of: the third and fourth attending medical professionals.

18. A method according to claim 14, wherein at least one of the attending medical professionals is at a remote location from where the colonoscopy is being performed.

19. A method according to claim 14, wherein colon visualization is performed during insertion and retraction of the colonoscope.

20. A colonoscope for use by a colonoscopy performing team, comprising: controls configured for simultaneous use by two attending medical professionals, wherein the respective controls for each attending medical professional are insufficient, alone, to control the colonoscope to perform a colonoscopy.
